(19) United States
(12) Patent Application Publication
(10) Pub. No.: US 2020/0268234 A1
(43) Pub. Date: Aug. 27, 2020

(54) OPTICAL-SCANNING OBSERVATION DEVICE AND OPTICAL-SCANNING-OBSERVATION-DEVICE OPERATION METHOD

(71) Applicant: OLYMPUS CORPORATION, Tokyo (JP)

(72) Inventors: Sanae Nakagawa, Tokyo (JP); Takeshi Mori, Tokyo (JP)

(73) Assignee: OLYMPUS CORPORATION, Tokyo (JP)

(21) Appl. No.: 15/931,989

(22) Filed: May 14, 2020

Related U.S. Application Data
(63) Continuation of application No. PCT/JP2017/042203, filed on Nov. 24, 2017.

(51) Int. Cl. A61B 1/00; H04N 5/225; A61B 5/00

(52) U.S. Cl. CPC A61B 1/00172; A61B 1/00009; A61B 5/0062; H04N 5/2256

(57) ABSTRACT

An optical-scanning observation device includes: a light source unit outputting illumination light; a light scanning unit scanning the illumination light; a light detection unit detecting signal light produced at each scan position and generating and outputting a light detection signal based on intensity of the signal light; a power supply unit supplying power to the light detection unit; a determination unit determining, every time the light detection signal is output, whether the intensity of the signal light is equal to or greater than a threshold set on the basis of a current capacity of the power supply unit; and an image generating unit generating an image having a pixel value based on the intensity of the signal light. The image generating unit generates an image having a saturated pixel value in a region determined that the intensity of the signal light is equal to or greater than the threshold.

### CROSS-REFERENCE TO RELATED APPLICATIONS

This is a continuation of International Application PCT/JP2017/042203 which is hereby incorporated by reference herein in its entirety.

### TECHNICAL FIELD

The present invention relates to an optical-scanning observation device and an optical-scanning-observation-device operation method.

### BACKGROUND ART

In the related art, there is a known optical-scanning observation device that spirally scans illumination light on an observation target and that acquires an image based on the brightness of reflected light from the observation target (for example, see PTL 1). In the optical-scanning observation device of PTL 1, when a brightness outside of a permissible range is detected at the rate of a threshold or greater during one frame period, the light intensity of illumination light for the next frame period is adjusted, thereby adjusting the brightness of an image, without relying on image processing.

### CITATION LIST

### Patent Literature

{PTL 1} Publication of Japanese Patent No. 5467756

### SUMMARY OF INVENTION

According to one aspect, the present invention provides an optical-scanning observation device including: a light source unit that outputs illumination light; a light scanning unit that scans the illumination light; a light detection unit that detects signal light produced, in a subject, at each scan position of the illumination light through irradiation of the illumination light and that generates and outputs a light detection signal based on intensity of the signal light; a power supply unit that supplies power to the light detection unit; a determination unit that determines, every time the light detection signal is output from the light detection unit, whether the intensity of the signal light, which is detected by the light detection unit, is equal to or greater than a prescribed acceptable threshold set on the basis of a current capacity of the power supply unit; and an image generating unit that generates an image having a pixel value based on the intensity of the signal light, wherein the image generating unit generates an image having a saturated pixel value in a region determined by the determination unit that the intensity of the signal light is equal to or greater than the prescribed acceptable threshold.

According to another aspect, the present invention provides an optical-scanning observation device including: a light source that outputs illumination light; a scanner that scans the illumination light; a light sensor that detects signal light produced, in a subject, at each scan position of the illumination light through irradiation of the illumination light and that generates and outputs a light detection signal based on intensity of the signal light; a power supply that supplies power to the light sensor; and a processor, wherein the processor determines, every time the light detection signal is output from the light sensor, whether the intensity of the signal light, which is detected by the light sensor, is equal to or greater than a prescribed acceptable threshold set on the basis of a current capacity of the power supply; and the processor generates an image having a saturated pixel value in a region determined that the intensity of the signal light is equal to or greater than the prescribed acceptable threshold.

According to still another aspect, the present invention provides an optical-scanning-observation-device operation method for an optical-scanning observation device that includes a light source unit that outputs illumination light, a light scanning unit that scans the illumination light, a light detection unit that detects signal light produced, in a subject, at each scan position of the illumination light through irradiation of the illumination light and that generates and outputs a light detection signal based on intensity of the signal light, and a power supply unit that supplies power to the light detection unit, the method including: determining, every time the light detection signal is output from the light detection unit, whether the intensity of the signal light, which is detected by the light detection unit, is equal to or greater than a prescribed acceptable threshold set on the basis of a current capacity of the power supply unit; and generating an image having a saturated pixel value in a region determined that the intensity of the signal light is equal to or greater than the prescribed acceptable threshold.

### BRIEF DESCRIPTION OF DRAWINGS

FIG. 1 is an overall configuration diagram of an optical-scanning observation device according to a first embodiment of the present invention.

FIG. 2 is a view for explaining the relationship between the level of a light detection signal and the quality of an image.

FIG. 3 is a flowchart showing the operation of the optical-scanning observation device shown in FIG. 1.

FIG. 4 is an overall configuration diagram of an optical-scanning observation device according to a second embodiment of the present invention.

FIG. 5 is a flowchart showing the operation of the optical-scanning observation device shown in FIG. 4.

FIG. 6 is an overall configuration diagram of a modification of the optical-scanning observation device shown in FIG. 4.

FIG. 7 is a flowchart showing the operation of an optical-scanning observation device shown in FIG. 6.

FIG. 8 is a flowchart showing the operation of an optical-scanning observation device according to a third embodiment of the present invention.

FIG. 9 is a continuation of the flowchart shown in FIG. 8.

### DESCRIPTION OF EMBODIMENTS

### First Embodiment

An optical-scanning observation device 100 according to a first embodiment of the present invention will be described below with reference to the drawings.

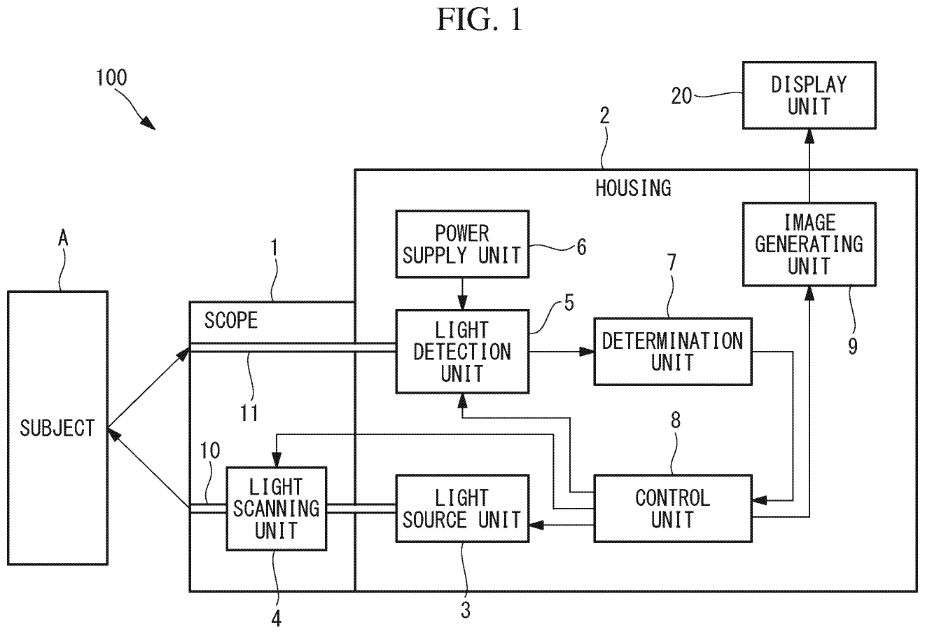

As shown in FIG. 1, the optical-scanning observation device 100 of this embodiment is an optical-scanning endoscope device that includes: a long scope 1 that is inserted into an observation target, such as the inside of a human body or an engine part of an aircraft; and a housing 2 that is connected to a proximal end of the scope 1. A display unit 20 that displays an image generated in the housing 2 is connected to the housing 2.

Furthermore, the optical-scanning observation device 100 includes: a light source unit 3 that outputs laser light (illumination light); a light scanning unit (scanner) 4 that scans laser light to be radiated onto a subject A; a light detection unit 5 that detects reflected light (signal light) of the laser light, coming from the subject A; a power supply unit (power supply) 6 that is connected to the light detection unit 5 and that supplies power to the light detection unit 5; a determination unit 7 that determines the intensity of the reflected light detected by the light detection unit 5; a control unit 8 that controls the light source unit 3, the light scanning unit 4, and the light detection unit 5; and an image generating unit 9 that generates a 2D image based on the intensity of reflected light. The light source unit 3, the light detection unit 5, the power supply unit 6, the determination unit 7, the control unit 8, and the image generating unit 9 are provided inside the housing 2, and the light scanning unit 4 is provided inside the scope 1.

The light source unit 3 includes laser light sources of red (R), green (G), and blue (B), such as laser diodes, and outputs laser light of R, G, and B simultaneously or in order. The laser light may be pulsed light or continuous light.

In the scope 1, an illumination optical fiber 10 that guides laser light from the light source unit 3 to a distal end section of the scope 1 is disposed in the longitudinal direction. Laser light supplied from the light source unit 3 to the optical fiber 10 is emitted from a distal end of the optical fiber 10 toward the subject A opposed to a distal end of the scope 1, and is radiated onto the subject A in the form of a spot.

The light scanning unit 4 includes a piezoelectric or electromagnetic actuator (not shown) provided at a distal end section of the optical fiber 10, and the actuator radially vibrates the distal end of the optical fiber 10, thereby scanning the laser light emitted from the distal end of the optical fiber 10.

The light scanning unit 4 may scan the laser light by using a galvanometer mirror or a MEMS (Micro Electro Mechanical Systems) mirror.

The light detection unit 5 is a light sensor such as an avalanche photodiode (APD). In the scope 1, a light-receiving optical fiber 11 that guides reflected light from the distal end of the scope 1 to the light detection unit 5 is disposed in the longitudinal direction. The light detection unit 5 detects reflected light that enters via the optical fiber 11 in synchronization with a sampling signal from the control unit 8, generates an analog light detection signal having the level corresponding to the intensity of the reflected light, and outputs the light detection signal. The output light detection signal is input to the determination unit 7.

An amplifier circuit that amplifies the light detection signal may be provided inside the light detection unit 5 or between the light detection unit 5 and the determination unit 7.

The light detection unit 5 generates a light detection signal by drawing, from the power supply unit 6, a current required to generate the light detection signal. The current drawn from the power supply unit 6 by the light detection unit 5 is increased as the intensity of reflected light detected by the light detection unit 5 becomes larger.

The power supply unit 6 has a normal range in which the magnitude of an output current is normal and an output limit that is greater than the current capacity, which is an upper limit of the normal range.

Here, in this embodiment, it is assumed that the subject A is an object containing a reflecting body that specularly reflects laser light. Thus, reflected light from the reflecting body includes specularly reflected light and diffusely reflected light. Specularly reflected light of laser light is very strong compared with diffusely reflected light. When the light detection unit 5 detects very strong specularly reflected light and draws, from the power supply unit 6, a current that exceeds the output limit, the power supply unit 6 has a function of temporarily reducing the output voltage in order to suppress the output of the current. A drop and rise in the output voltage at this time are slower than the scan speed of laser light scanned by the light scanning unit 4. Therefore, when the scanning trajectory has a spiral shape, the state in which the output voltage has dropped lasts over a plurality of laps.

The determination unit 7 determines whether the level of a light detection signal (i.e., the intensity of reflected light detected by the light detection unit 5) is equal to or greater than a prescribed first threshold (prescribed acceptable threshold) and outputs a determination result to the control unit 8 together with the light detection signal. Furthermore, after determining that the level of the light detection signal is equal to or greater than the prescribed first threshold, the determination unit 7 determines whether the level of a light detection signal is equal to or less than a prescribed second threshold, and outputs a determination result to the control unit 8 together with the light detection signal. The prescribed first threshold is a value set on the basis of the current capacity of the power supply unit 6, and the prescribed second threshold is a value less than the prescribed first threshold. The prescribed first threshold and the prescribed second threshold will be described later.

Although FIG. 1 shows the determination unit 7 as a function separate from the control unit 8, instead of this, the determination unit 7 may be realized as a part of the function of the control unit 8.

Furthermore, the light detection signal may be directly input to the control unit 8 from the light detection unit 5 without the intervening determination unit 7. In this case, only a determination result is output from the determination unit 7 to the control unit 8.

The control unit 8 sends a drive signal to the light scanning unit 4, thereby controlling scanning of laser light performed by the light scanning unit 4. The control unit 8 sends a sampling signal to the light detection unit 5 at prescribed sampling intervals, thereby controlling detection of reflected light performed by the light detection unit 5. Furthermore, the control unit 8 calculates, on the basis of the drive signal and the sampling signal, the laser-light irradiation position in the scanning trajectory at the time when the reflected light is detected by the light detection unit 5, and outputs, to the image generating unit 9, the calculated irradiation position in association with a pixel value, to be described later.

The control unit 8 controls the light intensity of laser light output by the light source unit 3 and generates a pixel value, on the basis of a determination result from the determination unit 7.

Specifically, during a normal time in which diffusely reflected light of laser light is produced in the subject A, the control unit 8 controls the light source unit 3 so as to output laser light having a light intensity at a prescribed initial light-intensity level. The prescribed initial light-intensity level is a light-intensity level at which the level of a light detection signal of diffusely reflected light becomes less than a saturation level. When the level of a light detection signal is equal to or greater than the saturation level, the pixel value is saturated.

Then, if the determination unit 7 determines that the level of a light detection signal is equal to or greater than the prescribed first threshold, the control unit 8 controls the light source unit 3 so as to reduce the light intensity of laser light to a prescribed low light-intensity level. The prescribed low light-intensity level is a light-intensity level that is less than the initial light-intensity level and at which the level of a light detection signal of specularly reflected light of laser light becomes less than the saturation level. For example, the low light-intensity level is set to a prescribed value, a prescribed fraction of the initial light-intensity level, or the level obtained by subtracting a prescribed value from the initial light-intensity level.

After the light intensity of laser light is reduced to the low light-intensity level, if the determination unit 7 determines that the level of a light detection signal is equal to or less than the prescribed second threshold, the control unit 8 controls the light source unit 3 so as to return the light intensity of laser light to the initial light-intensity level.

In order to reduce the light intensity of laser light, the control unit 8 may reduce all light intensities of the R, G, and B laser light or may turn off some of the R, G, and B laser light. For example, the control unit 8 may turn off the R and B laser light and turn on only the G laser light.

Alternatively, in order that the time integration of the light intensity of laser light during a certain period is reduced, it is also possible to turn on at least one color of laser light in a partial period during the certain period and to turn off every laser light in the remaining period during the certain period. In this case, because there is a timing at which a light detection signal is not output from the light detection unit 5 regardless of a sampling signal, the determination unit 7 intermittently performs determination.

The control unit 8 subjects a light detection signal received from the determination unit 7 to digital conversion by means of an analog-to-digital converter (not shown), thereby obtaining a digital value of the light detection signal. At this time, the digital value is limited within the range of values that the pixel value can take. Specifically, if the light detection signal is equal to or greater than the saturation level, the maximum value (saturation value) of the pixel value can be taken as the digital value, regardless of the level of the light detection signal.

When controlling the light intensity of laser light to be at the initial light-intensity level, the control unit 8 outputs, as the pixel value, the digital value of the light detection signal to the image generating unit 9. On the other hand, when controlling the light intensity of laser light to be at the low light-intensity level, the control unit 8 sends, as the pixel value, the saturation value to the image generating unit 9, regardless of the obtained digital value.

The image generating unit 9 generates an image by two-dimensionally arraying the pixel values, which are received from the control unit 8, on the basis of the irradiation positions. Therefore, the image generating unit 9 generates an image that has, in a region corresponding to a period during which laser light has the initial light-intensity level, pixel values according to the intensities of reflected light and that has saturated pixel values in a region corresponding to a period during which laser light has the low light-intensity level. The generated image is output from the image generating unit 9 to the display unit 20 and is displayed on the display unit 20.

Here, the relationship between the level of a light detection signal and the quality of an image generated by the image generating unit 9 will be described below.

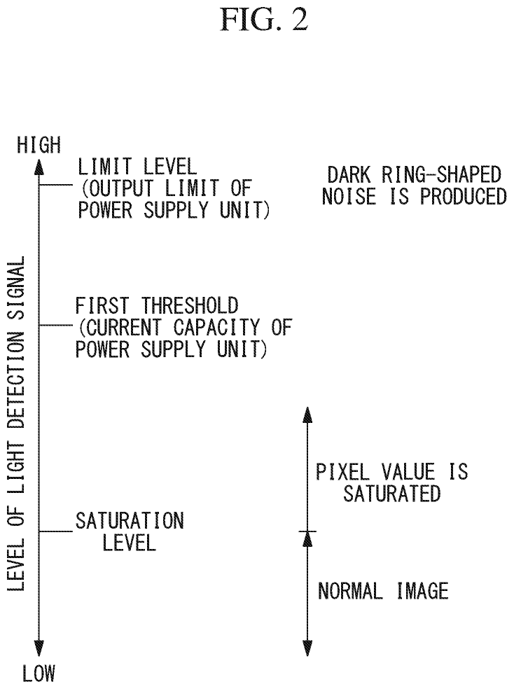

During the normal time, in which diffusely reflected light of laser light is produced in the subject A, as shown in FIG. 2, light detection signals become less than the saturation level, which corresponds to the maximum value (saturation value) of a pixel value, thus generating a normal image having pixel values that are obtained according to the levels of the light detection signals and that are less than the saturation value. On the other hand, when specularly reflected light of laser light is produced in the subject A, light detection signals become equal to or greater than the saturation level, thus generating an image having saturated pixel values.

When strong specularly reflected light that exceeds the limit level, which corresponds to the output limit of the power supply unit 6, is detected by the light detection unit 5, the current drawn from the power supply unit 6 to the light detection unit 5 exceeds the output limit. Immediately thereafter, the output voltage of the power supply unit 6 drops for a fixed period, thereby making light detection signals less for the fixed period, regardless of the intensities of reflected light entering the light detection unit 5. Thus, a region extending over a plurality of pixels and corresponding to the period during which the output voltage has dropped appears as dark noise in an image. When the scanning trajectory of laser light has a spiral shape, such a region becomes dark ring-shaped noise.

Here, the prescribed first threshold, which is used in the determination unit 7, is the level of a light detection signal that corresponds to the current capacity of the power supply unit 6.

The prescribed second threshold, which is used in the determination unit 7, is the level of a light detection signal that is less than the saturation level when it is assumed that the light intensity of laser light is returned from the low light-intensity level to the initial light-intensity level. Specifically, the prescribed second threshold is calculated on the basis of the following relational expression.

Initial light-intensity level:Level of light detection signal less than saturation level=Low light-intensity level:Prescribed second threshold

The above-described functions of the determination unit 7, the control unit 8, and the image generating unit 9 are realized when a processor, such as an arithmetic processing unit represented by a CPU, an FPGA, an LSI, and an ASIC, executes processing according to a program. Specifically, a processor (not shown) and a non-transitory storage medium (not shown) are provided inside the housing 2, and the program for causing the processor to execute the processing of the determination unit 7, the control unit 8, and the image generating unit 9 is stored in the storage medium.

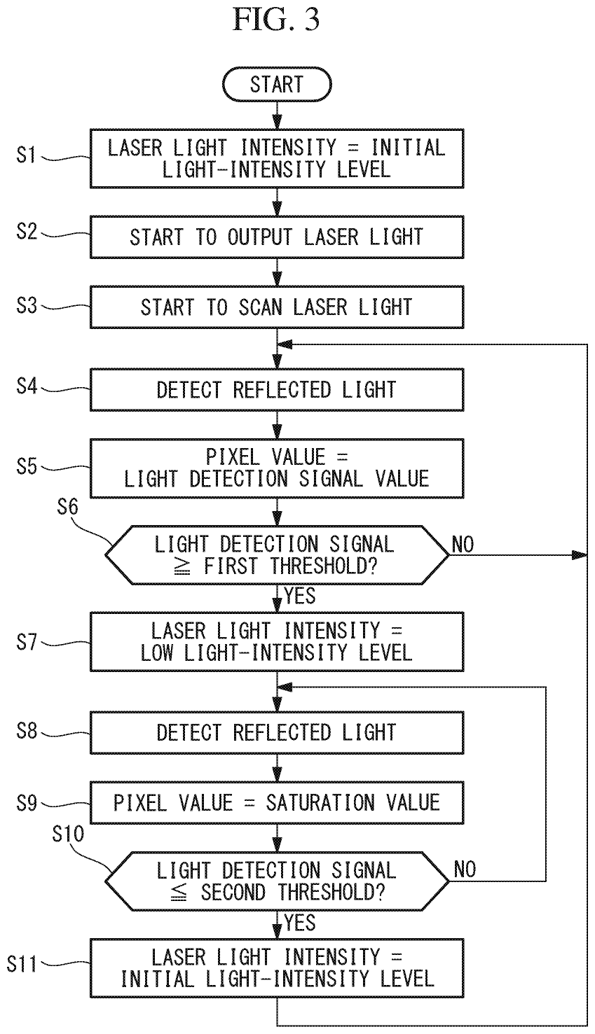

Next, the operation of the optical-scanning observation device 100 will be described below with reference to FIG. 3.

The control unit 8 makes the light source unit 3 start to output laser light at the initial light-intensity level (Steps S1 and S2) and also makes the light scanning unit 4 start to scan the laser light (Step S3). Accordingly, spot-like laser light is scanned on the subject A, and reflected light is produced at the irradiation position of the laser light. The reflected light is received at a distal end surface of the scope 1 by the optical fiber 11 and is guided to the light detection unit 5 by the optical fiber 11.

The control unit 8 makes the light detection unit 5 detect reflected light at the prescribed sampling intervals (Step S4). The light detection unit 5 outputs, every time reflected light is detected, a light detection signal to the control unit 8 via the determination unit 7, and the control unit 8 subjects the light detection signal to digital conversion, thus obtaining a pixel value (Step S5). The obtained pixel value is sent from the control unit 8 to the image generating unit 9 together with the irradiation position of the laser light. Next, in the image generating unit 9, the pixel values are arrayed on the basis of the irradiation positions, thus generating a 2D image. The generated image is displayed on the display unit 20.

Here, every time a light detection signal is output from the light detection unit 5 in Step S4, the determination unit 7 determines whether the level of the light detection signal is equal to or greater than the prescribed first threshold (Step S6). When the light detected by the light detection unit 5 is diffusely reflected light, the level of a light detection signal becomes less than the prescribed first threshold. If it is determined that the level of the light detection signal is less than the prescribed first threshold (NO in Step S6), the control unit 8 maintains the light intensity of laser light at the initial light-intensity level and repeats Steps S4 and S5.

On the other hand, when laser light is specularly reflected by a reflecting body, such as metal, present in the subject A, the light detection unit 5 detects strong specularly reflected light, and the level of a light detection signal becomes equal to or greater than the prescribed first threshold. If it is determined that the level of the light detection signal is equal to or greater than the prescribed first threshold (YES in Step S6), the control unit 8 reduces the light intensity of laser light from the initial light-intensity level to the low light-intensity level (Step S7), and, thereafter, makes the light detection unit 5 detect reflected light at prescribed sampling intervals, as in Step S4 (Step S8). Because reflected light produced in the subject A becomes weak due to the reduction in the light intensity of laser light, the level of a light detection signal is immediately reduced to be equal to or less than the saturation level, and the current drawn from the power supply unit 6 by the light detection unit 5 is reduced to be less than the current capacity.

While the light intensity of laser light is reduced to the low light-intensity level, the control unit 8 generates, as a pixel value, a saturation value regardless of the digital value of a light detection signal (Step S9). Therefore, in a generated image, irradiation positions of laser light whose light intensity has been reduced become a region in which the pixel values are saturated.

Every time a light detection signal is output from the light detection unit 5 in Step S8, the determination unit 7 determines whether the level of the light detection signal is equal to or less than the prescribed second threshold, i.e., whether the light detection signal is equal to or less than the saturation level even when the light intensity of laser light is returned to the initial light-intensity level (Step S10). The control unit 8 maintains the light intensity of laser light at the low light-intensity level until it is determined in Step S10 that a light detection signal is equal to or less than the prescribed second threshold. If it is determined in Step S10 that a light detection signal is equal to or less than the prescribed second threshold, the control unit 8 returns the light intensity of laser light to the initial light-intensity level (Step S11). Thereafter, Steps S4 to S11 are repeated.

In this way, according to this embodiment, every time a light detection signal is output from the light detection unit 5, it is determined whether strong reflected light corresponding to the current capacity is produced, on the basis of the level of the light detection signal. Then, if it is determined that strong reflected light is produced, before the light detection unit 5 detects the next reflected light, the light intensity of laser light is immediately reduced such that the level of a light detection signal is reduced to be equal to or less than the saturation level. In this way, in rapid response to the fact that strong reflected light is produced, the level of a light detection signal is adjusted on a light-detection-signal basis, thereby offering an advantage in that it is possible to prevent very strong reflected light that corresponds to the output limit of the power supply unit 6 from being produced and to prevent dark noise from being produced in an image.

Furthermore, by reducing the light intensity of laser light when strong reflected light is produced, there is an advantage in that the current consumption can be reduced.

Furthermore, in an image, by saturating pixel values in a region corresponding to a period during which the light intensity of laser light is suppressed to the low light-intensity level, there is an advantage in that an observer can easily recognize the region in which strong specularly reflected light is produced, on the basis of the saturated pixel values.

In this embodiment, although the prescribed first threshold, which is used in the determination unit 7, is the level of a light detection signal that corresponds to the current capacity of the power supply unit 6, the prescribed first threshold may also be another value that is set on the basis of the current capacity of the power supply unit 6, as long as the current drawn from the power supply unit 6 by the light detection unit 5 when specularly reflected light is produced can be controlled to be less than the current capacity. Specifically, the prescribed first threshold can be set to an arbitrary value within the range from the saturation level to the limit level, which corresponds to the output limit of the power supply unit 6.

For example, the prescribed first threshold may also be the level of a light detection signal that corresponds to about 90% of the current capacity in view of individual differences in the performance of the power supply unit 6 or may also be the level of a light detection signal that corresponds to about 50% of the current capacity in order to prevent a load from being applied to the power supply unit 6.

Alternatively, when the rated value of the current of the light detection unit 5 is less than the current capacity of the power supply unit 6, the prescribed first threshold may be the rated value of the light detection unit 5. In this case, it is possible to prevent an excessive load from being applied to the light detection unit 5.

In this embodiment, although the timing at which the light intensity of laser light is returned from the low light-intensity level to the initial light-intensity level is determined on the basis of the level of a light detection signal, instead of this, the timing may be determined on the basis of a pixel value.

In this case, the prescribed second threshold is a value at which a pixel value becomes less than the saturation value when it is assumed that the light intensity of laser light is returned from the low light-intensity level to the initial light-intensity level. In Step S10, the determination unit 7 determines whether the pixel value generated by the control unit 8 is equal to or less than the prescribed second threshold.

By doing so, it is also possible to appropriately determine the timing at which the light intensity of laser light is returned from the low light-intensity level to the initial light-intensity level, after specularly reflected light is produced.

In this embodiment, although the determination unit 7 determines the intensity of reflected light detected by the light detection unit 5, on the basis of the level of a light detection signal, instead of this, the determination may be made on the basis of an output current from the power supply unit 6.

As described above, the magnitude of a current drawn from the power supply unit 6 by the light detection unit 5 is determined depending on the intensity of reflected light detected by the light detection unit 5. Therefore, whether the intensity of reflected light detected by the light detection unit 5 is equal to or greater than the prescribed first threshold can be determined on the basis of an output current from the power supply unit 6.

In this embodiment, although the light intensity of laser light is reduced to the low light-intensity level at which a light detection signal becomes less than the saturation level, when the level of a light detection signal is equal to or greater than the prescribed first threshold, instead of this, the light intensity of laser light may also be reduced to a low light-intensity level that is equal to or greater than the saturation level.

By doing so, in the period during which the light intensity of laser light is reduced to the low light-intensity level, as in the period during which the light intensity of laser light is the initial light-intensity level, the digital value of a light detection signal can be used as a pixel value, thus making it possible to eliminate the processing for converting the digital value to the saturation value.

### Second Embodiment

Next, an optical-scanning observation device 200 according to a second embodiment of the present invention will be described below with reference to the drawings.

In this embodiment, configurations different from those in the first embodiment will be described, identical reference signs are given to configurations common to those in the first embodiment, and a description thereof will be omitted.

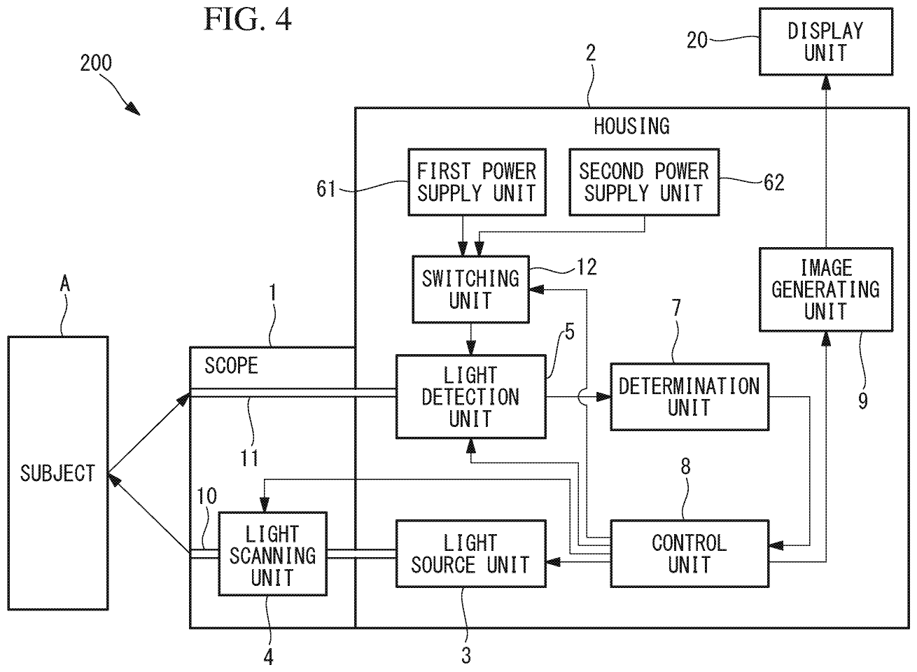

As shown in FIG. 4, the optical-scanning observation device 200 of this embodiment includes a first power supply unit 61, a second power supply unit 62, and a switching unit 12, in addition to the scope 1, the housing 2, the light source unit 3, the light scanning unit 4, the light detection unit 5, the determination unit 7, the control unit 8, and the image generating unit 9.

The first power supply unit 61 and the second power supply unit 62 are connected to the light detection unit 5 in parallel, via the switching unit 12. The first power supply unit 61 supplies a first voltage to the light detection unit 5. The second power supply unit 62 supplies a second voltage that is lower than the first voltage to the light detection unit 5.

The switching unit 12 selectively connects one of the first power supply unit 61 and the second power supply unit 62 to the light detection unit 5, according to control performed by the control unit 8. The switching unit 12 is formed of, for example, a field effect transistor (FET), a bipolar transistor, or an analog switch.

The light detection unit 5 is, for example, an APD and has a function of multiplying a light detection signal in accordance with a voltage supplied thereto. Specifically, compared with when the first power supply unit 61 is connected to the light detection unit 5, when the second power supply unit 62 is connected to the light detection unit 5, the multiplication factor becomes small, and a light detection signal also becomes less. Therefore, a current drawn from the second power supply unit 62 by the light detection unit 5 is lower than a current drawn from the first power supply unit 61 by the light detection unit 5.

Here, the first voltage makes the level of a light detection signal of diffusely reflected light less than the saturation level. The second voltage is lower than the first voltage and makes the level of a light detection signal of specularly reflected light of laser light less than the saturation level.

The determination unit 7 determines whether the level of a light detection signal is equal to or greater than the prescribed first threshold, and outputs a determination result to the control unit 8 together with the light detection signal. Furthermore, after determining that the level of the light detection signal is equal to or greater than the prescribed first threshold, the determination unit 7 determines whether the level of a light detection signal is equal to or less than the prescribed second threshold, and outputs a determination result to the control unit 8 together with the light detection signal.

As in the first embodiment, the control unit 8 performs control of the light scanning unit 4 and the light detection unit 5 and calculation of an irradiation position.

Furthermore, the control unit 8 controls the switching unit 12 and generates a pixel value on the basis of the determination result from the determination unit 7.

Specifically, during the normal time, the control unit 8 controls the switching unit 12 so as to connect the first power supply unit 61 to the light detection unit 5. Then, if the determination unit 7 determines that the light detection signal is equal to or greater than the prescribed first threshold, the control unit 8 controls the switching unit 12 so as to connect the second power supply unit 62 to the light detection unit 5. After the second power supply unit 62 is connected to the light detection unit 5, if a light detection signal becomes equal to or less than the prescribed second threshold, the control unit 8 controls the switching unit 12 so as to connect the first power supply unit 61 to the light detection unit 5 again.

Here, the prescribed first threshold is the level of a light detection signal that corresponds to the current capacity of the first power supply unit 61.

The prescribed second threshold is the level of a light detection signal that is less than the saturation level when it is assumed that the current supplied to the light detection unit 5 is returned from the second voltage to the first voltage. Specifically, the prescribed second threshold is calculated on the basis of the following relational expression.

Multiplication factor in the light detection unit 5 when the first voltage is supplied thereto:Level of a light detection signal less than the saturation level=Multiplication factor in the light detection unit 5 when the second voltage is supplied thereto:Prescribed second threshold

When the first power supply unit 61 is connected to the light detection unit 5, the control unit 8 outputs, as a pixel value, the digital value of a light detection signal to the image generating unit 9. On the other hand, when the second power supply unit 62 is connected to the light detection unit 5, the control unit 8 sends, as a pixel value, the saturation value to the image generating unit 9 regardless of the obtained digital value.

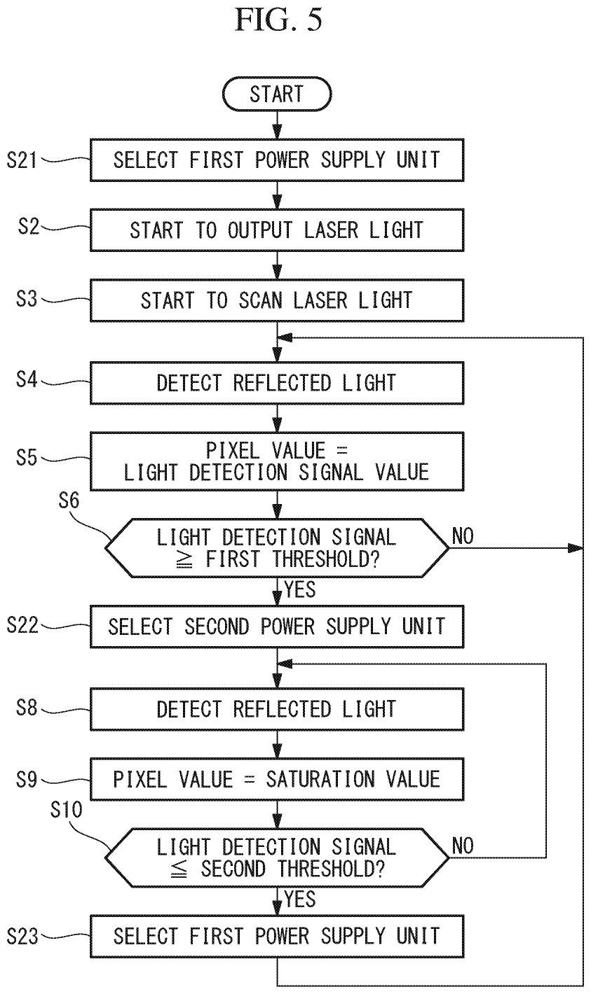

Next, the operation of the optical-scanning observation device 200 will be described below with reference to FIG. 5.

The control unit 8 controls the switching unit 14 so as to connect the first power supply unit 61 to the light detection unit 5 (Step S21), makes the light source unit 3 start to output laser light (Step S2), and makes the light scanning unit 4 start to scan the laser light (Step S3). Then, the control unit 8 makes the light detection unit 5 detect reflected light at prescribed sampling intervals (Step S4) and subjects a light detection signal to digital conversion, thus obtaining a pixel value (Step S5). Furthermore, every time a light detection signal is output from the light detection unit 5 in Step S4, the determination unit 7 determines whether the level of the light detection signal is equal to or greater than the prescribed first threshold (Step S6).

If it is determined that the light detection signal is less than the prescribed first threshold (NO in Step S6), the control unit 8 maintains the connection of the first power supply unit 61 to the light detection unit 5 and repeats Steps S4 and S5. On the other hand, if it is determined that the light detection signal is equal to or greater than the prescribed first threshold (YES in Step S6), the control unit 8 controls the switching unit 14 so as to connect the second power supply unit 62 to the light detection unit 5 (Step S22) and then makes the light detection unit 5 detect reflected light at prescribed sampling intervals (Step S8). Due to the reduction in the voltage supplied to the light detection unit 5, the level of a light detection signal is immediately reduced to be equal to or less than the saturation level, and the current drawn from the second power supply unit 62 by the light detection unit 5 becomes less than the current capacity.

Every time a light detection signal is output from the light detection unit 5 in Step S8, the determination unit 7 determines whether the light detection signal is equal to or less than the prescribed second threshold (Step S10). The control unit 8 maintains the connection of the second power supply unit 62 to the light detection unit 5 until it is determined in Step S10 that a light detection signal is equal to or less than the prescribed second threshold. If it is determined in Step S10 that a light detection signal is equal to or less than the prescribed second threshold, the control unit 8 controls the switching unit 14 so as to connect the first power supply unit 61 to the light detection unit 5 again (Step S23). Hereinafter, Steps S4 to S6, S22, S8 to S10, and S23 are repeated.

In this way, according to this embodiment, every time a light detection signal is output from the light detection unit 5, it is determined whether strong reflected light that corresponds to the current capacity is produced, on the basis of the level of the light detection signal. Then, if it is determined that strong reflected light is produced, before the light detection unit 5 detects the next reflected light, the voltage to be supplied to the light detection unit 5 is immediately suppressed so as to reduce the level of a light detection signal to be equal to or less than the saturation level. In this way, in rapid response to the fact that strong reflected light is produced, the level of a light detection signal is adjusted on a light-detection-signal basis, thereby offering an advantage in that it is possible to prevent very strong reflected light that corresponds to the output limit of the first power supply unit 61 from being produced and to prevent dark noise from being produced in an image.

Furthermore, by reducing the voltage to be supplied to the light detection unit 5 when strong reflected light is produced, there is an advantage in that the current consumption can be reduced.

Furthermore, in an image, by saturating pixel values in a region corresponding to a period during which the voltage to be supplied to the light detection unit 5 is suppressed to the second voltage, there is an advantage in that an observer can easily recognize the region in which strong specularly reflected light is produced, on the basis of the saturated pixel values.

In this embodiment, although the voltage to be supplied to the light detection unit 5 is suppressed in order to reduce the level of the light detection signal, which is generated and output by the light detection unit 5, instead of this, it is also possible to suppress the current flowing from the power supply unit 6 to the light detection unit 5.

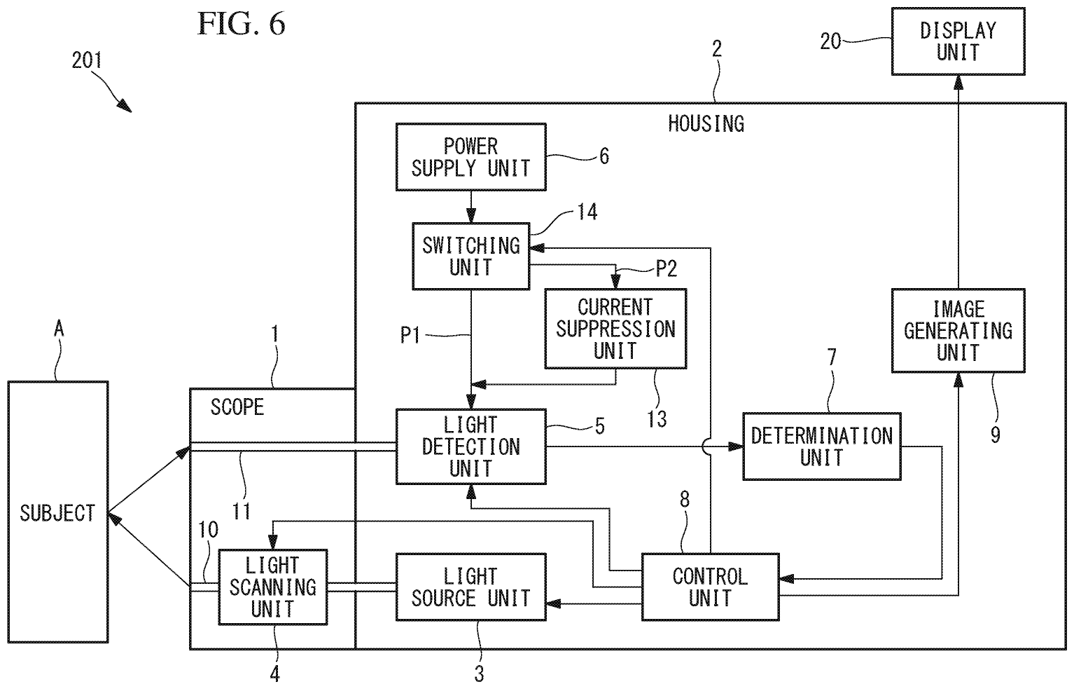

Specifically, as shown in FIG. 6, it is also possible to include: a current suppression unit 13 that is provided between the power supply unit 6 and the light detection unit 5; and a switching unit 14 that switches enable/disable of the current suppression unit 13.

The current suppression unit 13 is configured to flow a current that is less than the current capacity of the power supply unit 6 and not to flow a current that is equal to or greater than the current capacity of the power supply unit 6. The current suppression unit 13 is formed of, for example, any one of a transistor, a resistor, and an operational amplifier or a circuit obtained by using a combination thereof. The current suppression unit 13 may suppress the current to about 90% of the current capacity in view of individual differences in the performance of the power supply unit 6 or may suppress the current to about 50% of the current capacity in order to prevent a load from being applied to the power supply unit 6.

The switching unit 14 is formed of, for example, a field effect transistor (FET), a bipolar transistor, or an analog switch. In the example shown in FIG. 6, a first path P1 in which the power supply unit 6 is directly connected to the light detection unit 5 and a second path P2 in which the power supply unit 6 is connected to the light detection unit 5 via the current suppression unit 13 are formed, and the switching unit 14 is configured to disable the current suppression unit 13 by selecting the first path P1 and to enable the current suppression unit 13 by selecting the second path P2. Instead of this, the switching unit 14 may be provided inside the current suppression unit 13.

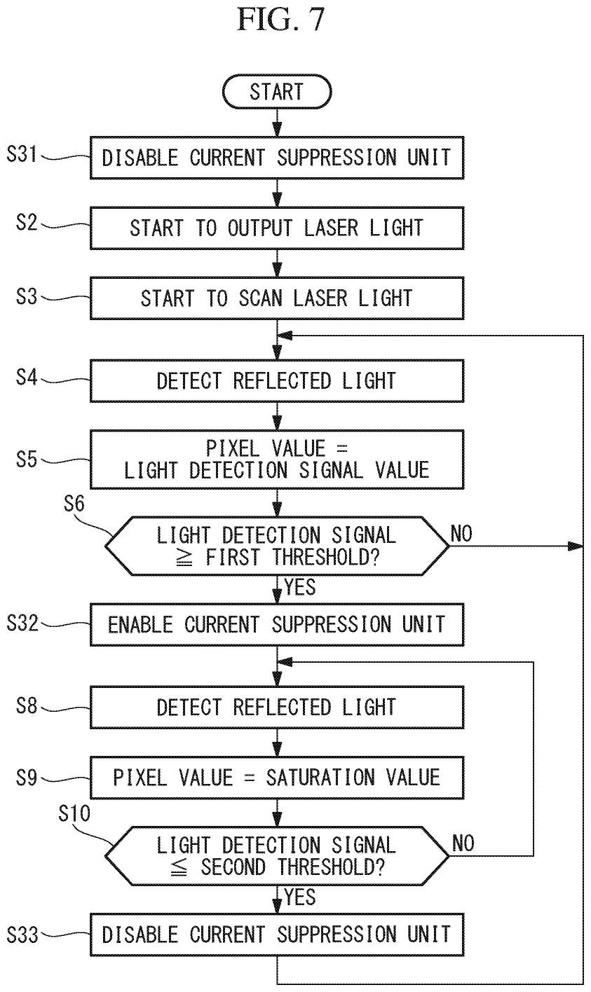

As shown in FIG. 7, the control unit 8 controls the switching unit 14 so as to disable the current suppression unit 13 during the normal time (Step S31). Then, if the determination unit 7 determines that a light detection signal is equal to or greater than the prescribed first threshold (YES in Step S6), the control unit 8 controls the switching unit 14 so as to enable the current suppression unit 13 (Step S32). Accordingly, the level of the light detection signal, which is generated and output by the light detection unit 5, is reduced, thus preventing the light detection unit 5 from drawing a current that is equal to or greater than the current capacity, from the power supply unit 6. After the current suppression unit 13 is switched to be enabled, when the level of a light detection signal becomes equal to or less than the prescribed second threshold (YES in Step S10), the control unit 8 controls the switching unit 14 so as to disable the current suppression unit 13 (Step S33).

In the modification shown in FIG. 6, the prescribed second threshold is the level of a light detection signal that is equal to or greater than the saturation level. Therefore, the digital value of the light detection signal obtained in Step S9 becomes the saturation value.

Alternatively, the prescribed second threshold may also be the level of a light detection signal that is less than the saturation level.

In this way, by suppressing the current drawn to the light detection unit 5, the level of a light detection signal is adjusted on a light-detection-signal basis such that the light detection signal becomes less than the level corresponding to the current capacity, thus making it possible to prevent the output current of the power supply unit 6 from reaching the output limit. Furthermore, by enabling the current suppression unit 13 only when strong reflected light enters the light detection unit 5, it is possible to suppress the power consumption compared with a case in which the current suppression unit 13 is always enabled, and to reduce noise generated at the circuit.

### Third Embodiment

Next, an optical-scanning observation device according to a third embodiment of the present invention will be described below with reference to the drawings.

In this embodiment, configurations different from those in the first and second embodiments will be described, identical reference signs are given to configurations common to those in the first and second embodiments, and a description thereof will be omitted.

The device configuration of the optical-scanning observation device of this embodiment is the same as the device configuration of an optical-scanning observation device 201 shown in FIG. 6. However, this embodiment differs from the first and second embodiments in that the control unit 8 performs both the control of the light source unit 3, which is performed in the first embodiment, and the control of the switching unit 14, which is performed in the second embodiment.

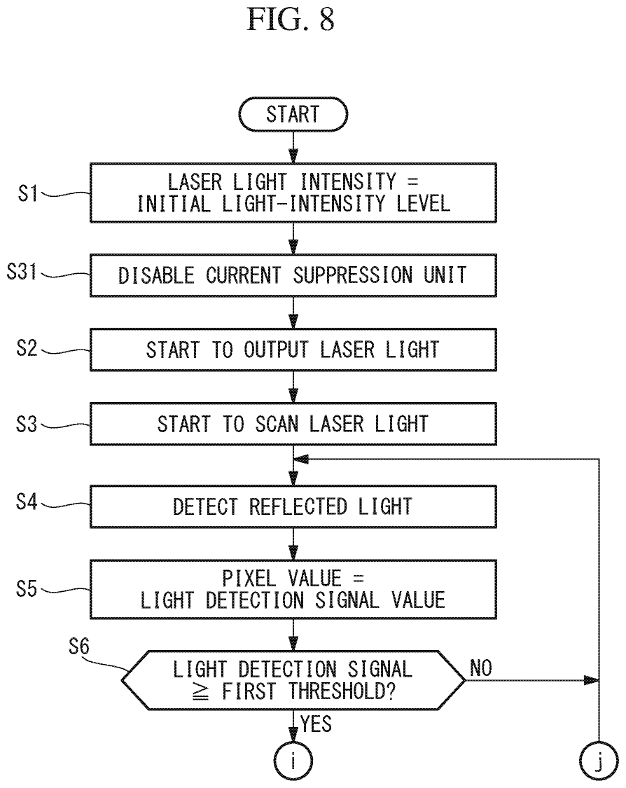

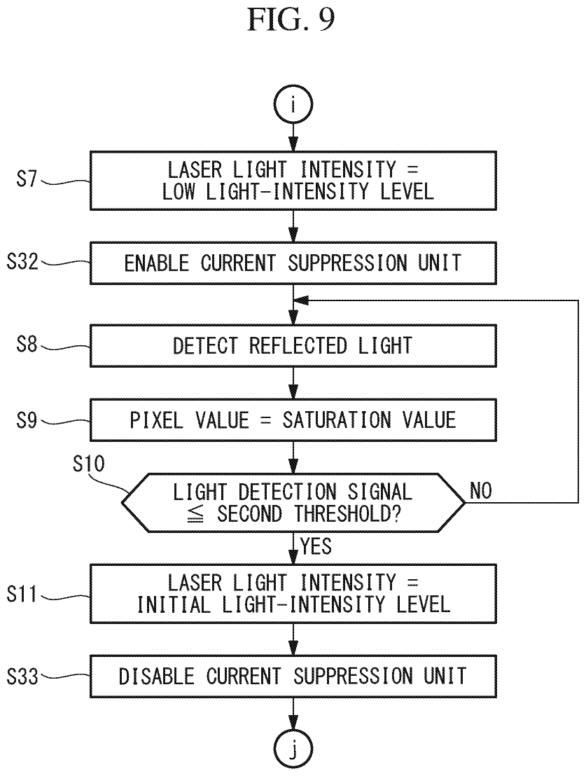

Specifically, as shown in FIGS. 8 and 9, the control unit 8 controls the switching unit 14 to disable the current suppression unit 13 (Step S31), makes the light source unit 3 start to output laser light at the initial light-intensity level (Steps S1 and S2), and makes the light scanning unit 4 start to scan the laser light (Step S3). Steps S4 to S6 are the same as those in the first embodiment.

If it is determined that the level of a light detection signal is less than the prescribed first threshold (NO in Step S6), the control unit 8 maintains the light intensity of laser light at the initial light-intensity level, maintains the connection of the first power supply unit 61 to the light detection unit 5, and repeats Steps S4 and S5. On the other hand, if it is determined that the level of the light detection signal is equal to or greater than the prescribed first threshold (YES in Step S6), the control unit 8 reduces the light intensity of laser light from the initial light-intensity level to the low light-intensity level (Step S7) and controls the switching unit 14 to enable the current suppression unit 13 (Step S32).

Then, if it is determined in Step S10 that the level of a light detection signal is equal to or less than the prescribed second threshold, the control unit 8 returns the light intensity of laser light to the initial light-intensity level (Step S11) and controls the switching unit 14 to disable the current suppression unit 13 (Step S33). The second threshold may be the second threshold used in the first embodiment or may be the second threshold used in the modification of the second embodiment.

According to this embodiment, in addition to the advantageous effects in the first and second embodiments, the following advantageous effects are afforded. By performing suppression of the light intensity of laser light and suppression of the current to the light detection unit 5 in combination, there is an advantage in that it is possible to reliably reduce the light detection signal to be equal to or less than the saturation level, even with respect to very strong reflected light that cannot be handled only through suppression of the light intensity of laser light or only through suppression of the current to the light detection unit 5. Furthermore, because the response speed of a reduction in the light intensity of laser light is faster than the response speed of a reduction in the current performed by the current suppression unit 13, there is an advantage in that a light detection signal can be rapidly reduced, compared with a case in which only the current suppression unit 13 is used.

The above-described embodiment also leads to the following aspects.

According to one aspect, the present invention provides an optical-scanning observation device including: a light source unit that outputs illumination light; a light scanning unit that scans the illumination light; a light detection unit that detects signal light produced, in a subject, at each scan position of the illumination light through irradiation of the illumination light and that generates and outputs a light detection signal based on the intensity of the signal light; a power supply unit that supplies power to the light detection unit; a determination unit that determines, every time the light detection signal is output from the light detection unit, whether the intensity of the signal light, which is detected by the light detection unit, is equal to or greater than a prescribed acceptable threshold set on the basis of a current capacity of the power supply unit; and a control unit that suppresses, in response to determining by the determination unit that the intensity of the signal light is equal to or greater than the prescribed acceptable threshold, at least one of the light intensity of the illumination light and the power supplied from the power supply unit to the light detection unit.

According to this aspect, when illumination light radiated from the light source unit onto the subject is scanned on the subject by the light scanning unit, signal light produced at each scan position of the subject is detected by the light detection unit, and the light detection unit draws a current corresponding to the intensity of the signal light from the power supply unit, thus generating a light detection signal, and outputs the light detection signal. The light detection signals are associated with the irradiation positions of the illumination light, thus making it possible to acquire an image of the subject.

In this case, every time the light detection signal is output from the light detection unit, the determination unit determines whether the intensity of the signal light, which is detected by the light detection unit, is equal to or greater than the prescribed acceptable threshold, which is set on the basis of the current capacity of the power supply unit. Then, if it is determined that the intensity of the signal light is equal to or greater than the prescribed acceptable threshold, the control unit suppresses at least one of the light intensity of the illumination light and power supplied to the light detection unit, thereby reducing the level of a light detection signal to be generated next, such that the current drawn from the power supply unit by the light detection unit becomes appropriate with respect to the current capacity. In this way, the level of the light detection signal can be adjusted on a light-detection-signal basis, in rapid response to the fact that strong signal light is produced.

In the above-described aspect, the power supply unit may include a first power supply unit and a second power supply unit that are selectively connected to the light detection unit; the first power supply unit may supply a first voltage to the light detection unit; the second power supply unit may supply a second voltage lower than the first voltage to the light detection unit; and the control unit may switch, in response to determining by the determination unit that the intensity of the signal light is equal to or greater than the prescribed acceptable threshold, the power supply unit connected to the light detection unit, from the first power supply unit to the second power supply unit.

As the voltage supplied to the light detection unit is increased, the level of the light detection signal, which is generated by the light detection unit, is increased, and the current drawn from the power supply unit by the light detection unit is also increased. Therefore, by switching the power supply unit connected to the light detection unit from the first power supply unit to the second power supply unit, the level of a light detection signal can be reduced.

The above-described aspect may further include a current suppression unit that suppresses a current flowing from the power supply unit to the light detection unit, wherein the control unit may make, in response to determining by the determination unit that the intensity of the signal light is equal to or greater than the prescribed acceptable threshold, the current suppression unit suppress the current supplied from the power supply unit to the light detection unit.

In this way, the current flowing into the light detection unit when the light detection unit draws the current from the power supply unit is suppressed by the current suppression unit, thereby making it possible to reduce the level of a light detection signal.

The above-described aspect may further include an image generating unit that generates an image having a pixel value based on the intensity of the signal light, wherein the image generating unit may generate an image having a saturated pixel value when the control unit suppresses at least one of the light intensity of the illumination light and power supplied from the power supply unit to the light detection unit.

Furthermore, the above-described aspect may further include an image generating unit that generates an image having a pixel value based on the intensity of the signal light, wherein the control unit may output, to the image generating unit, the pixel value based on the light detection signal and output, to the image generating unit, a saturated pixel value when the control unit suppresses at least one of the light intensity of the illumination light and power supplied from the power supply unit to the light detection unit.

By doing so, in an image generated by the image generating unit, a region where strong signal light is produced is expressed as a region where the pixel values are saturated. Therefore, an observer can recognize the region where strong signal light is produced.

According to another aspect, the present invention provides an optical-scanning observation device including: a light source that outputs illumination light; a scanner that scans the illumination light; a light sensor that detects signal light produced, in a subject, at each scan position of the illumination light through irradiation of the illumination light and that generates and outputs a light detection signal based on the intensity of the signal light; a power supply that supplies power to the light sensor; and a processor, wherein the processor determines, every time the light detection signal is output from the light sensor, whether the intensity of the signal light, which is detected by the light sensor, is equal to or greater than a prescribed acceptable threshold set on the basis of a current capacity of the power supply; and the processor suppresses, in response to determining that the intensity of the signal light is equal to or greater than the prescribed acceptable threshold, at least one of the light intensity of the illumination light and power supplied from the power supply to the light sensor.

According to still another aspect, the present invention provides an optical-scanning-observation-device operation method for an optical-scanning observation device that includes a light source unit that outputs illumination light, a light scanning unit that scans the illumination light, a light detection unit that detects signal light produced, in a subject, at each scan position of the illumination light through irradiation of the illumination light and that generates and outputs a light detection signal based on the intensity of the signal light, and a power supply unit that supplies power to the light detection unit, the method including: determining, every time the light detection signal is output from the light detection unit, whether the intensity of the signal light, which is detected by the light detection unit, is equal to or greater than a prescribed acceptable threshold set on the basis of a current capacity of the power supply unit; and suppressing, in response to determining that the intensity of the signal light is equal to or greater than the prescribed acceptable threshold, at least one of the light intensity of the illumination light and power supplied from the power supply unit to the light detection unit.

This still another aspect may further include generating an image having a saturated pixel value in response to determining that the intensity of the signal light is equal to or greater than the prescribed acceptable threshold.

### REFERENCE SIGNS LIST

100, 200, 201 optical-scanning observation device
1 scope
2 housing
3 light source unit (light source)
4 light scanning unit (scanner)
5 light detection unit (light sensor)
6 power supply unit (power supply)
7 first power supply unit (power supply)
8 second power supply unit (power supply)
9 determination unit (processor)
10 control unit (processor)
11 image generating unit (processor)
10, 11 optical fiber
12, 14 switching unit
13 current suppression unit
A subject

1. An optical-scanning observation device comprising:
a light source unit that outputs illumination light;
a light scanning unit that scans the illumination light;
a light detection unit that detects signal light produced, in a subject, at each scan position of the illumination light through irradiation of the illumination light and that generates and outputs a light detection signal based on intensity of the signal light;
a power supply unit that supplies power to the light detection unit;
a determination unit that determines, every time the light detection signal is output from the light detection unit, whether the intensity of the signal light, which is detected by the light detection unit, is equal to or greater than a prescribed acceptable threshold set on the basis of a current capacity of the power supply unit; and
an image generating unit that generates an image having a pixel value based on the intensity of the signal light,
wherein the image generating unit generates an image having a saturated pixel value in a region determined by the determination unit that the intensity of the signal light is equal to or greater than the prescribed acceptable threshold.

2. The optical-scanning observation device according to claim 1, further comprising a control unit that suppresses, in response to determining by the determination unit that the intensity of the signal light is equal to or greater than the prescribed acceptable threshold, at least one of the light intensity of the illumination light and the power supplied from the power supply unit to the light detection unit,
wherein the image generating unit generates the image having the saturated pixel value in a region corresponding to a period during which at least one of the light intensity of the illumination light and the power supplied from the power supply unit to the light detection unit is suppressed by the control unit.

3. The optical-scanning observation device according to claim 2,
wherein the power supply unit comprises a first power supply unit and a second power supply unit that are selectively connected to the light detection unit;
the first power supply unit supplies a first voltage to the light detection unit;
the second power supply unit supplies a second voltage lower than the first voltage to the light detection unit; and
the control unit switches, in response to determining by the determination unit that the intensity of the signal light is equal to or greater than the prescribed acceptable threshold, the power supply unit connected to the light detection unit, from the first power supply unit to the second power supply unit.

4. The optical-scanning observation device according to claim 2, further comprising a current suppression unit that suppresses a current flowing from the power supply unit to the light detection unit,
wherein the control unit makes, in response to determining by the determination unit that the intensity of the signal light is equal to or greater than the prescribed acceptable threshold, the current suppression unit suppress the current supplied from the power supply unit to the light detection unit.

5. The optical-scanning observation device according to claim 2,
wherein the control unit outputs, to the image generating unit, a pixel value based on the light detection signal, and
wherein the control unit outputs, to the image generating unit, the saturated pixel value during at least one of the light intensity of the illumination light and the power supplied from the power supply unit to the light detection unit is suppressed by the control unit, regardless of a value of the light detection signal.

6. The optical-scanning observation device according to claim 2,
wherein the determination unit determines whether the intensity of the signal light, which is detected by the light detection unit, is equal to or less than a second threshold after the control unit suppresses at least one of the light intensity of the illumination light and the power supplied from the power supply unit to the light detection unit, the second threshold being less by a prescribed value than the prescribed acceptable threshold, and
wherein the control unit suppresses at least one of the light intensity of the illumination light and the power supplied from the power supply unit to the light detection unit until it is determined by the determination unit that the intensity of the signal light is equal to or less than the second threshold.

7. An optical-scanning observation device comprising:
a light source that outputs illumination light;
a scanner that scans the illumination light;
a light sensor that detects signal light produced, in a subject, at each scan position of the illumination light through irradiation of the illumination light and that generates and outputs a light detection signal based on intensity of the signal light;
a power supply that supplies power to the light sensor; and
a processor,
wherein the processor determines, every time the light detection signal is output from the light sensor, whether the intensity of the signal light, which is detected by the light sensor, is equal to or greater than a prescribed acceptable threshold set on the basis of a current capacity of the power supply, and
the processor generates an image having a saturated pixel value in a region determined that the intensity of the signal light is equal to or greater than the prescribed acceptable threshold.

8. The optical-scanning observation device according to claim 7, wherein the processor suppresses, in response to determining that the intensity of the signal light is equal to or greater than the prescribed acceptable threshold, at least one of the light intensity of the illumination light and the power supplied from the power supply to the light sensor, and generates the image having the saturated pixel value in a region corresponding to a period during which at least one of the light intensity of the illumination light and the power supplied from the power supply to the light sensor is suppressed.

9. An optical-scanning-observation-device operation method for an optical-scanning observation device that includes a light source unit that outputs illumination light, a light scanning unit that scans the illumination light, a light detection unit that detects signal light produced, in a subject, at each scan position of the illumination light through irradiation of the illumination light and that generates and outputs a light detection signal based on intensity of the signal light, and a power supply unit that supplies power to the light detection unit, the method comprising:
determining, every time the light detection signal is output from the light detection unit, whether the intensity of the signal light, which is detected by the light detection unit, is equal to or greater than a prescribed acceptable threshold set on the basis of a current capacity of the power supply unit; and
generating an image having a saturated pixel value in a region determined that the intensity of the signal light is equal to or greater than the prescribed acceptable threshold.

10. The optical-scanning-observation-device operation method according to claim 9, wherein in the generating, suppressing, in response to determining that the intensity of the signal light is equal to or greater than the prescribed acceptable threshold, at least one of the light intensity of the illumination light and the power supplied from the power supply unit to the light detection unit, and generating the image having the saturated pixel value in a region corresponding to a period during which at least one of the light intensity of the illumination light and the power supplied from the power supply unit to the light detection unit is suppressed.
